(19) United States
(12) Patent Application Publication
(10) Pub. No.: US 2015/0105829 A1
(43) Pub. Date: Apr. 16, 2015

(54) BONE FIXATION DEVICE

(71) Applicant: Austofix Group Limited, North Plympton (AU)

(72) Inventor: Ewen Laird, North Plympton (AU)

(73) Assignee: AUSTOFIX GROUP LIMITED, North Plympton, SA (AU)

(21) Appl. No.: 14/403,160

(22) PCT Filed: May 22, 2013
(86) PCT No.: PCT/AU2013/000536

§ 371 Date: Nov. 21, 2014

(30) Foreign Application Priority Data
May 22, 2012 (AU) 2012902106

(51) Int. Cl. A61B 17/86; A61B 17/80

(52) U.S. Cl. CPC A61B 17/863; A61B 17/809
USPC 606/315

(57) ABSTRACT

A bone fixation element (100) for fixing a bone fixation plate (200) to a bone (5) is disclosed. The bone fixation element (100) includes a shank (140) receivable into the bone (5) and a head (120) securable to the bone fixation plate (200). The head (120) has an external locking thread (160) including a first thread section (166) adapted to progressively deform a portion of a hole (220) in the bone fixation plate (200) as the head (120) of the bone fixation element (100) is inserted into the hole (220). The external locking thread (160) further includes a second substantially parallel thread section (164) adapted to engage with a portion of the hole (220) deformed by the first thread section (166) to thereby secure the head (120) of the bone fixation element (100) to the bone fixation plate (200). The bone fixation element (100) may be a variable angle locking screw.

### PRIORITY DOCUMENTS

The present application claims priority from Australian Provisional Patent Application No. 2012902106 titled “Bone Fixation Device” and filed on 22 May 2012, the entire contents of which is hereby incorporated by reference in its entirety.

### TECHNICAL FIELD

The present invention relates to the field of bone fixation systems, and in particular to bone fixation elements such as pegs and screws for use in fixing a bone fixation plate to a bone.

### BACKGROUND

Internal fracture fixation is often necessary when a fracture occurs within the human body. A goal of the fixation is to provide immediate stabilization to the fracture. Another goal is to encourage rapid healing and return mobility to an individual thus preventing muscular atrophy. Metallic plates (CoCrMo alloy, stainless steel or titanium) can be fixed to the outside of the bone using a variety of fixation elements such as screws, pins, nails, pegs and wires, selectively placed around a fracture site.

Many current bone fixation systems use locking screws to fix the plate to the bone. Some plates use fixed angle locking screws, which are fixed in a particular orientation relative to the plate, whilst other plates use variable angle locking screws that can be fixed to the bone within a range of angles, relative to the plate. There are various ways of allowing a screw to be inserted at variable angles, but many conventional variable angle screws have a tapered ‘locking’ thread, which engages with an internal thread of a hole in the plate. The variable angle screw can be inserted at a surgeon-directed angle to the plate, which will usually result in the screw cross-threading in the screw hole, and deforming the internal thread of the screw hole. The range of permissible angles can vary, but may be, for example, ±15 degrees.

Fixed angle locking screws are generally considered to provide increased stability, relative to variable angle screws. Variable angle screws can suffer from lack of stability from some directions, and typically also have a relatively low back-out torque, such that if the screw is loosened by a fraction of a turn, the grip of the male thread is lost and the screw can begin to toggle in the hole.

On the other hand, the advantage of a variable angle screw is that it allows the surgeon to better tailor the application of the plate system to the specific nature of the bone fracture.

It would be desirable to provide a locking screw that allowed for variable angle insertion, but provided increased stability relative to tapered variable angle screws.

### SUMMARY

According to a first aspect, there is provided a bone fixation element for fixing a bone fixation plate to a bone, including:

a shank receivable into the bone;

a head securable to the bone fixation plate, the head having an external locking thread including:

a first thread section adapted to progressively deform a portion of a hole in the bone fixation plate as the head of the bone fixation element is inserted into the hole; and

a second substantially parallel thread section adapted to engage with a portion of the hole deformed by the first thread section to thereby secure the head of the bone fixation element to the bone fixation plate.

In one form, the first thread section starts at a first diameter and transitions into the second substantially parallel thread section having a second diameter that is greater than the first diameter.

In one form, the first thread section is tapered.

In one form, the external locking thread is a triple start thread.

In one form, the bone fixation element is a screw having a shank with a bone engaging thread.

In one form, a lead of the external locking thread is the same as a lead of the bone engaging thread.

In one form, the bone fixation element is a peg.

According to a second aspect, there is provided a bone fixation system for a bone, including:

a bone fixation plate;

a bone fixation element for fixing the bone fixation plate to the bone, including:

a shank receivable into the bone;

a head securable to the bone fixation plate, the head having an external locking thread including:

a first thread section adapted to progressively deform a portion of a hole in the bone fixation plate as the head of the bone fixation element is inserted into the hole; and

a second substantially parallel thread section adapted to engage with a portion of the hole deformed by the first thread section to thereby secure the head of the bone fixation element to the bone fixation plate.

In one form, the hole in the bone fixation plate has an internal thread which is progressively deformed by the first thread section of the external locking thread on the head of the bone fixation element.

In one form, the hole in the bone fixation plate has a series of concentric rings that are progressively deformed by the first thread section of the external locking thread on the head of the bone fixation element.

In one form, the bone fixation element has a greater material hardness than the bone fixation plate.

According to a third aspect, there is provided a bone fixation element for fixing a bone fixation plate to a bone, comprising:

an external locking thread, for engaging within a hole of the bone fixation plate, wherein the locking thread comprises:

a tapered thread section, for deforming an internal structure of the hole as the tapered thread section is screwed into the hole, so as to form engagement portions around the internal circumference of the hole; and

a substantially parallel thread section, for engaging with the engagement portions.

The engagement portions may be considered as an internal thread of the hole, formed by the tapered thread section. The use of a tapered thread allows the surgeon to direct the screw at an angle, and also helps to draw the screw into the plate, allowing the surgeon to form this ‘internal thread’ gradually, without requiring excessive axial loading. However, the engagement between the substantially parallel thread section and this ‘internal thread’ is able to provide a strong, stable locking function, which is significantly more stable than that offered by conventional tapered variable angle screws.

A wide variety of different internal structures could be used. However, the internal structure of the hole will typically be an internal parallel thread—this may allow the plate to also be used with conventional fixed angle screws, which could be inserted into the same hole. Preferably, the internal thread has a fine pitch, which helps to enable the formation of more engagement portions. However, some embodiments of the present invention may utilise concentric rings around the internal circumference of the hole, which could also be deformed by the tapered thread section so as to form the engagement portions for engagement with the parallel section of the locking thread.

The bone fixation element may be a screw, but in other embodiments may be a peg. It will typically comprise a head and a shaft (or shank), with the locking thread located near or on the head. In the case of a screw, a bone engagement thread (for engaging with the bone) will typically run along the majority of the length of the shaft. For screws, the locking thread preferably has the same lead as the bone engaging thread.

The locking thread may take various forms within the scope of the present invention, and may have different pitches in different embodiments of the invention. Preferably, the locking thread is a triple start thread, although locking threads having a different number of starts may be used depending on factors such as the materials used for the screw and the plate.

The tapered thread section is preferably tapered at a steeper angle than the maximum tilt intended for the screw in the plate.

According to a fourth aspect, there is provided a bone fixation system for a bone, comprising:

a bone fixation plate, having a hole with an internal structure; and

a bone fixation element to fix the bone fixation plate to the bone, the bone fixation element comprising an external locking thread, for engaging within the hole of the bone fixation plate, and the locking thread comprising:
a tapered thread section, for deforming the internal structure of the hole as the tapered thread section is screwed into the hole, so as to form engagement portions around the internal circumference of the hole; and
a substantially parallel thread section, for engaging with the engagement portions.

According to a fifth aspect, there is provided a bone fixation screw comprising:

a head;

a shaft having a bone engaging thread for engaging a bone; and

a triple start locking thread near the head of the screw, having a lead that is substantially the same as a lead of the bone engaging thread.

According to a sixth aspect, there is provided is provided a bone fixation system for a bone, comprising:

a bone fixation plate, having a hole with an internal structure; and

a bone fixation screw to fix the bone fixation plate to the bone, the bone fixation screw comprising:

a head;

a shaft having a bone engaging thread for engaging a bone; and

a triple start locking thread near the head of the screw, having a lead that is substantially the same as a lead of the bone engaging thread.

### BRIEF DESCRIPTION OF DRAWINGS

Embodiments of the present invention will be discussed with reference to the accompanying drawings wherein:

FIG. 1 is a schematic cross-section illustrating the basic use of a bone fixation element;

FIG. 2 is a side view of a variable angle screw according to an embodiment of the present invention;

FIG. 3 is a detailed view of the head and upper shank of the screw of FIG. 2;

FIGS. 4a-4c show, sequentially, the use of the screw of FIG. 2 with a bone fixation plate;

FIG. 5 is a cross-sectional perspective view of a hole in a bone fixation plate, for receiving the screw of FIG. 2;

FIG. 6 is a further detailed view of a screw according to an embodiment of the present invention;

FIG. 7 is a top view of the screw of FIG. 6;

FIG. 8 is a schematic view of a hole in a bone fixation plate showing the portions of the hole deformed by the first thread section that engage with the parallel thread section; and

FIG. 9 is a perspective view of a bone fixation system showing a variable angle screw being driven through a bone fixation plate and into a bone.

In the following description, like reference characters designate like or corresponding parts throughout the figures.

### DESCRIPTION OF EMBODIMENTS

Referring now to FIG. 1 there is shown a bone fixation element 100 engaged within a hole 220 in a bone fixation plate 200. The hole 220 has a parallel internal thread 225, and more details of this in the context of the present invention will be described later in this specification. As shown in FIG. 1, the bone fixation element 100 is engaged at a slight angle to the normal axis 20 of the bone-contacting surface 230 of the plate 200. The bone fixation element 100 is required to withstand loads (F) from any direction while at any allowable angle of orientation with respect to the bone fixation plate 200. Angle β represents the obtuse angle formed between the longitudinal axis 10 of the bone fixation element 100 and the plate 200 while angle α represents the acute angle formed between the longitudinal axis 10 of the bone fixation element 100 and the plate 200.

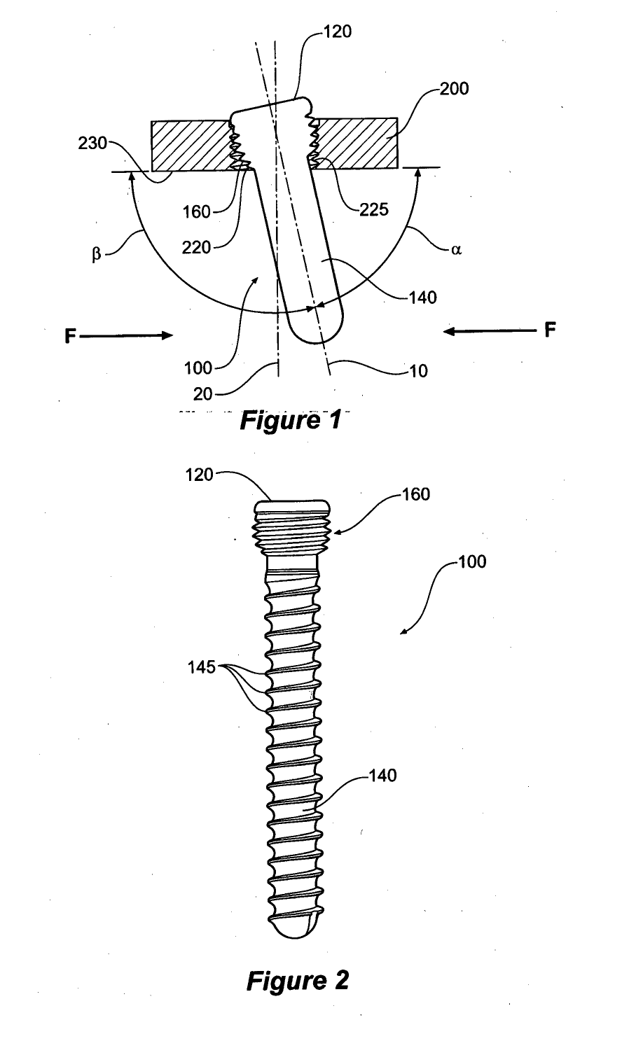

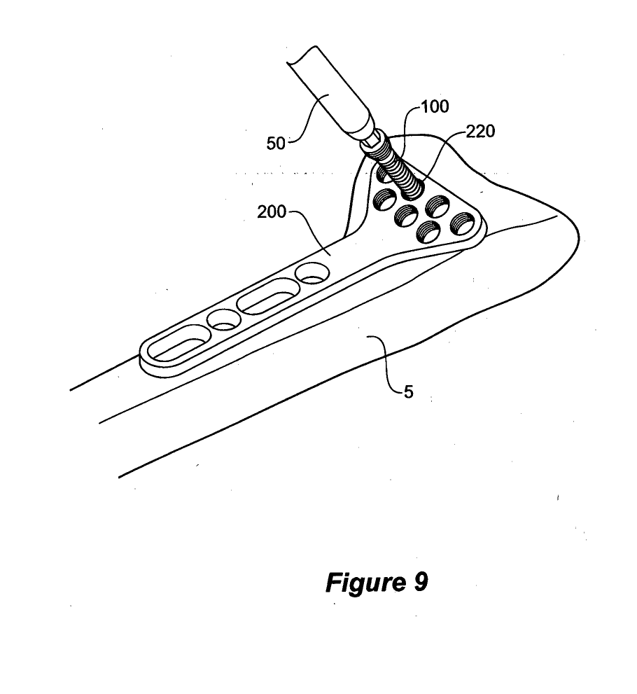

In a preferred embodiment, the bone fixation element 100 is a variable angle screw as shown in FIG. 2. The screw 100 comprises a head 120 and a shaft or shank 140 receivable into a bone. The head 120 has a slot or socket (not shown) enabling it to be driven by a tool 50 as shown for example in FIG. 9 where a screw 100 is shown being driven into plate 200 which is positioned to stabilise a fracture of a bone 5. Referring again to FIG. 2, the screw 100 further includes an external bone engaging thread 145 located along the shank 140, to engage the screw 100 with a bone, in order to fix a bone fixation plate 200 to the bone.

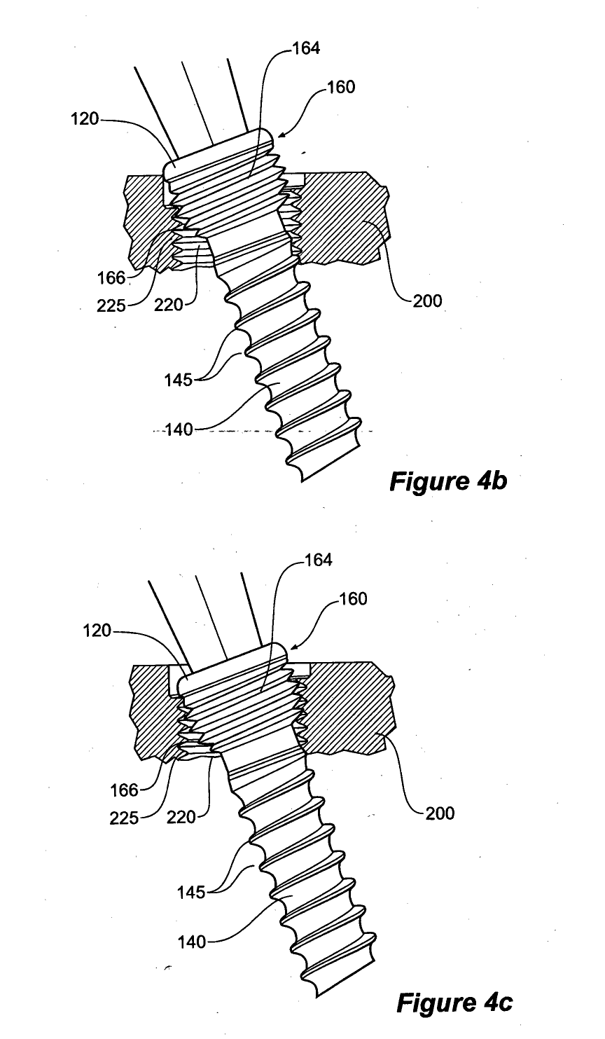

The head 120 of the screw 100 is securable to the bone fixation plate 200 and is provided with an external locking thread 160. The locking thread 160 is provided to engage with an internal thread 225 of a hole 220 in a bone fixation plate 200 (as shown in FIGS. 4a-4c).

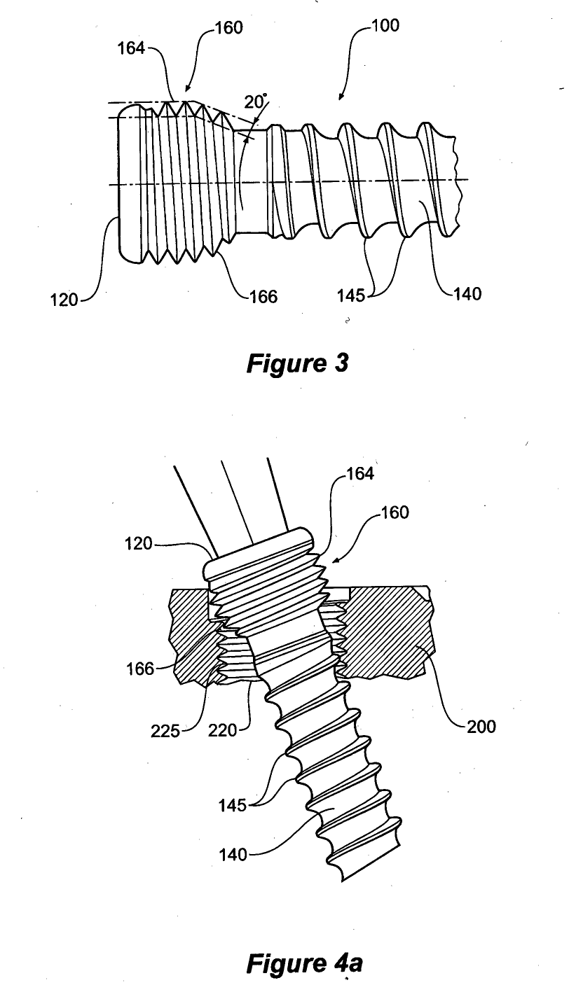

FIG. 3 depicts, in more detail, the locking thread 160 of the screw 100. The locking thread includes a first thread section 166 and a second substantially parallel thread section 164, located nearest the top of the screw head 120. The first thread section 166 may be a tapered thread section as shown in FIGS. 2 and 3, whereby the first thread section 166 is tapered at a 20 degree angle with respect to the shank 140. Preferably, the tapered thread section 166 is tapered at a steeper angle than the maximum tilt intended for the screw 100 in the plate 200. Accordingly, the screw 100 of this embodiment of the present invention would be most suitable for angles up to, for example, 15 degrees.

It will be understood that the features of the locking thread 160 may vary in different embodiments of the invention depending on a number of factors, including the desired angulation of the screw, the material of the screw and plate, and the lead or pitch of the cortical bone engaging thread of the screw.

In one embodiment, the locking thread 160 is a triple start locking thread that has the same lead as the bone engaging thread 145 on the shank 140. For example, with reference to FIG. 6, the locking thread 160 is triple start with a 1.2 mm lead, and the bone engaging thread 145 is double start with a 1.2 mm lead. With the two threads having the same lead, the screw progresses into the plate and bone at the same speed. This helps to avoid jamming during insertion, before the screw is fully seated.

Furthermore, in this embodiment, the parallel thread section 164 of the locking thread 160 is 0.5 mm long. As the crest to crest distance between locking threads is 0.4 mm, a triple lobe effect is generated as shown in FIG. 7. There are three equally spaced portions of locking thread 164 where two fully formed threads are generated at the full diameter. This provides additional stability to the screw at a wide variety of insertion angles.

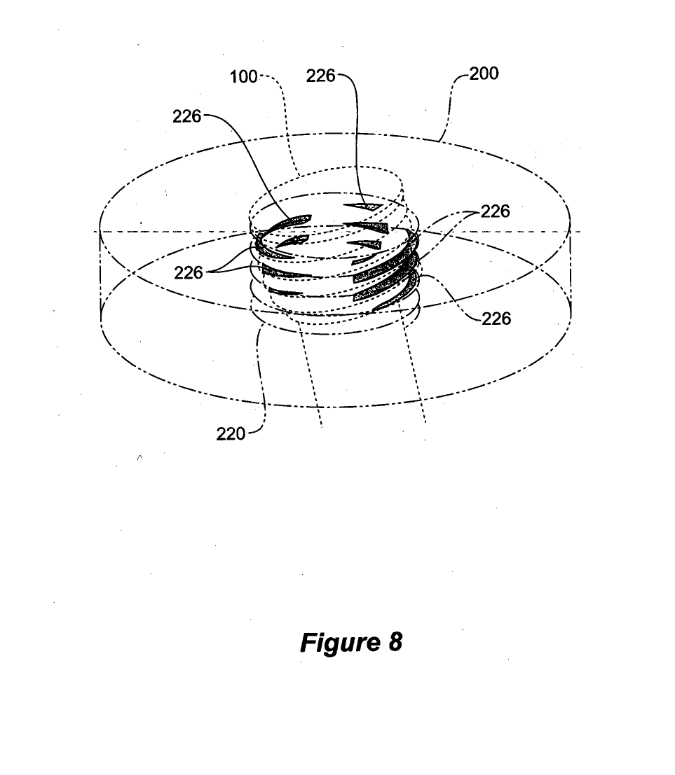

FIGS. 4a-4c depict the screw 100 of the present invention in use, in combination with a bone fixation plate 200. Initially, the screw 100 is inserted through the hole 220 and the bone engagement thread 145 is screwed into the bone. Once the screw 100 is sufficiently inserted, the external locking thread 160 begins to come into engagement with the internal thread 225 (or other internal structure) of the hole 220. The first thread section 166 is adapted to progressively deform a portion (internal structure such as an internal thread 225) of the hole 220 in the bone fixation plate 200 as the head 120 of the screw 100 is inserted into the hole 220. The second substantially parallel thread section 164 is adapted to engage with a portion of the hole 220 deformed by the first thread section 166 (i.e. the deformed internal thread represented by engagement portions 226 as shown in FIG. 8) to thereby secure the head 120 of the screw 100 to the bone fixation plate 200.

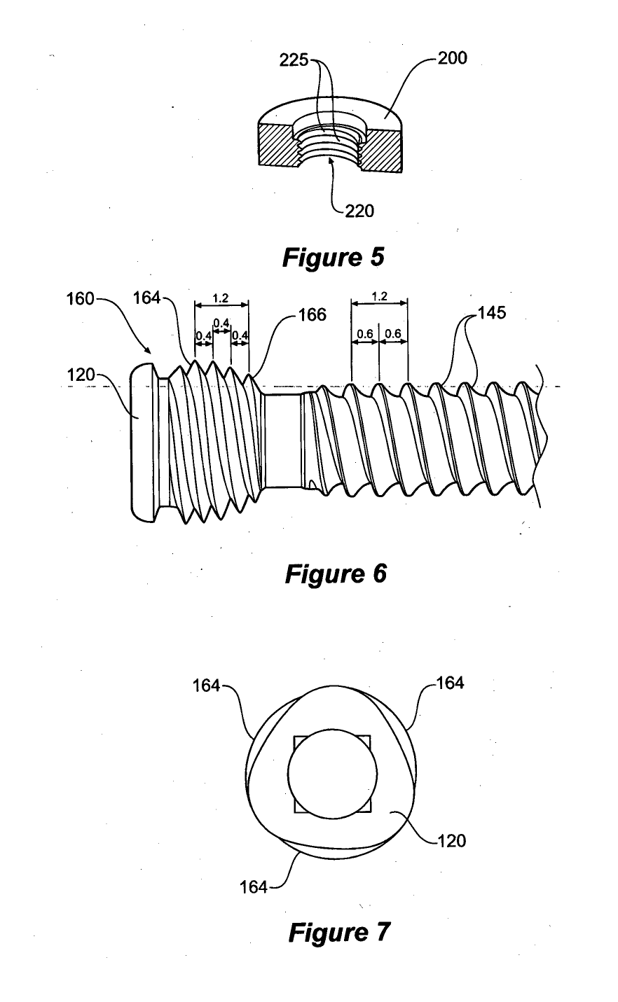

The internal thread 225, in this embodiment, is a twin start parallel thread, having two threads of equal lead starting at 180 degrees diametrically opposed. The vertical distance between each groove of the internal thread 225 is half the thread lead (where lead=pitch×number of threads). FIG. 5 depicts the internal thread 225 in more detail. However, it may be possible, in other embodiments, to use other internal structures (e.g. series of concentric rings, variable pitch single start threads, single start threads with a significantly different pitch than the pitch of the screw, threads of opposite hand to the screw) around the inner circumference of the hole 220.

The tapered thread section 166 is the first to come into contact with the internal thread 225 of the hole 220, as shown in FIG. 4a. This is a “lead-in” section, which steers the screw 100 into the internal thread 225, and “aligns” the locking thread 160 with the internal thread 225. The tapered section 166 assists by dragging the screw 100 into the internal thread 225, thus reducing the requirement for excessive axial loading to get the threads started.

FIG. 4b depicts the thread forming (deformation) stage in the use of the screw 100 according to this embodiment of the present invention. As the screw 100 is dragged into the internal thread 225 by rotation of the screw 100, the tapered section 166 begins to cross thread and deform the internal thread 225. As the tapered section 166 progresses, a new thread gradually develops, which comprises segments of the deformed original internal thread 225. This new deformed internal thread effectively provides engagement portions 226 (as shown in FIG. 8) around the inner circumference of the hole 200.

In this embodiment, as the locking thread and bone engaging thread are the same lead, there is no shearing of the plate threads as may sometimes be experienced in other designs with a differential of leads, causing the generation of burrs. This may be a greater problem where the screw is made of harder materials—e.g. CoCr.

As the screw 100 is inserted further into the hole 220, the parallel thread section 164 engages with the engagement portions 226 formed by the tapered thread section 166. Once the screw 100 is seated, there are a number of parallel threads engaged (of the parallel thread section 164 of the locking thread 160), as shown in FIG. 4c. The engagement of the parallel thread section 164 provides an effective stabilising effect on the screw, regardless of angulation.

Preferably, the tapered thread section 166 starts at a first diameter as shown for example in FIG. 3 and transitions into the parallel thread section 164 having a second diameter that is greater than the first diameter.

Accordingly, the screw 100 according to the present invention provides a significant stability advantage over conventional variable angle screws. The screw 100 of this embodiment of the present invention is capable of withstanding loads from various directions, due to the engagement of the parallel thread section 164 of the locking thread 160 with the engagement portions 226 formed by deforming an internal structure (e.g. internal thread 225) of a hole 220. When a conventional variable angle screw comprising a simple tapered thread is cross threaded into a parallel female thread, the screw contact on the obtuse angle aspect of the plate is reduced (compared to a fixed angle screw) to the extent that the screw is far less stable when loaded from the acutely angled side of the screw or from a wide angle either side of this direction. The variable angle screw of the present invention advantageously provides improved stability over variable angle screws known in the art.

Furthermore, because the screw 100 according to this embodiment of the present invention comprises a locking thread 160 with a parallel thread section 164, it requires a much higher backout torque. For conventional variable angle screws with tapered threads, these screws can begin to toggle in the hole immediately, once the tapered thread has loosened by a fraction of a turn. However, a screw 100 according to the above-described embodiment of the present invention will behave similarly to a normally engaged parallel thread, in that even if the screw is slightly loosened, there is sufficient engagement to prevent unwanted screw movement.

The parallel thread section 164 is considered, in this embodiment, to be precisely parallel. However, the present invention may be implemented in screws or pegs where this section 164 is not precisely parallel, but is still sufficiently close to parallel to provide the substance of the above advantages.

With regard to the material of the screw 100, the material of the screw 100 should generally be harder than the material of the plate 200, in order to create the new thread once the screw 100 is inserted. This also helps to prevent galling and cold welding of the threads.

By way of non-limiting examples, the following combinations of plate and screw materials may be used:

| Plate Material | Screw Material |
|---|---|
| Ti- grade 4 | Ti6AlV4 |
| 316LVM Stainless Steel | High Ni SS (ASTM 5832-9) |
| Ti6AlV4 | CoCr |

Furthermore, it is recommended that the crests of the locking thread 160 be relatively sharp, to assist in the thread forming operation and avoid the creation of break-away burrs. This also reduces the required insertion torque.

In relation to the screw head 120, rounding of the screw head 120 helps to reduce the possibility of soft tissue irritation when sitting slightly proud of the plate. The screw head 120 is also preferably of a sufficient size to act as a form of abutment that prevents the screw 100 from travelling completely through the screw hole 220 when the angulation approaches 0 degrees.

Tolerances of the locking thread and the internal thread on the plate are also important. Too much interference and the screws will require too much torque to engage, which could damage the screw driver or jam the screws. If there is insufficient interference, the threads will strip without fully locking onto the plate.

Throughout the specification and the claims that follow, unless the context requires otherwise, the words “comprise” and “include” and variations such as “comprising” and “including” will be understood to imply the inclusion of a stated integer or group of integers, but not the exclusion of any other integer or group of integers.

The reference to any prior art in this specification is not, and should not be taken as, an acknowledgement of any form of suggestion that such prior art forms part of the common general knowledge.

It will be appreciated by those skilled in the art that the invention is not restricted in its use to the particular application described. Neither is the present invention restricted in its preferred embodiment with regard to the particular elements and/or features described or depicted herein. It will be appreciated that the invention is not limited to the embodiment or embodiments disclosed, but is capable of numerous rearrangements, modifications and substitutions without departing from the scope of the invention as set forth and defined by the following claims.

1. A bone fixation element for fixing a bone fixation plate to a bone, including:
a shank receivable into the bone;
a head securable to the bone fixation plate, the head having an external locking thread including:
a first thread section adapted to progressively deform a portion of a hole in the bone fixation plate as the head of the bone fixation element is inserted into the hole; and
a second substantially parallel thread section adapted to engage with a portion of the hole deformed by the first thread section to thereby secure the head of the bone fixation element to the bone fixation plate.

2. The bone fixation element of claim 1 wherein the first thread section starts at a first diameter and transitions into the second substantially parallel thread section having a second diameter that is greater than the first diameter.

3. The bone fixation element of claim 1 or 2 wherein the first thread section is tapered.

4. The bone fixation element of any one of the preceding claims wherein the external locking thread is a triple start thread.

5. The bone fixation element of any one of the preceding claims wherein the bone fixation element is a screw having a shank with a bone engaging thread.

6. The bone fixation element of claim 5 wherein a lead of the external locking thread is the same as a lead of the bone engaging thread.

7. The bone fixation element of any one of claims 1 to 4 wherein the bone fixation element is a peg.

8. A bone fixation system for a bone, including:
a bone fixation plate;
a bone fixation element for fixing the bone fixation plate to the bone, including:
a shank receivable into the bone;
a head securable to the bone fixation plate, the head having an external locking thread including:
a first thread section adapted to progressively deform a portion of a hole in the bone fixation plate as the head of the bone fixation element is inserted into the hole; and
a second substantially parallel thread section adapted to engage with a portion of the hole deformed by the first thread section to thereby secure the head of the bone fixation element to the bone fixation plate.

9. The bone fixation system of claim 8 wherein the hole in the bone fixation plate has an internal thread which is progressively deformed by the first thread section of the external locking thread on the head of the bone fixation element.

10. The bone fixation system of claim 8 wherein the hole in the bone fixation plate has a series of concentric rings that are progressively deformed by the first thread section of the external locking thread on the head of the bone fixation element.

11. The bone fixation system of any one of claims 8 to 10 wherein the bone fixation element has a greater material hardness than the bone fixation plate.

12. The bone fixation system of any one of claims 8 to 11 wherein the first thread section starts at a first diameter and transitions into the second substantially parallel thread section having a second diameter that is greater than the first diameter.

13. The bone fixation system of any one of claims 8 to 12 wherein the first thread section is tapered.

14. The bone fixation system of any one of claims 8 to 13 wherein the external locking thread is a triple, start thread.

15. The bone fixation system of any one of claims 8 to 14 wherein the bone fixation element is a screw having a shank with a bone engaging thread.

16. The bone fixation system of any one of claims 8 to 15 wherein a lead of the external locking thread is the same as a lead of the bone engaging thread.
